(19) United States
(12) Patent Application Publication
(10) Pub. No.: US 2007/0053870 A1
(43) Pub. Date: Mar. 8, 2007

(54) Polysaccharide-functionalized nanoparticle, drug delivery system for controlled release comprising the same and preparation method thereof

(75) Inventors: Giyoong Tae, Gwangju (KR); Yong-Il Chung, Gwangju (KR)

Correspondence Address: ROTHWELL, FIGG, ERNST & MANBECK, P.C., 1425 K STREET, N.W., SUITE 800, WASHINGTON, DC, 20005, US

(73) Assignee: Gwangju Institute of Science and Technology, Gwangju (KR)

(21) Appl. No.: 11/391,480

(22) Filed: Mar. 29, 2006

(30) Foreign Application Priority Data
Sep. 8, 2005 (KR) 10-2005-0083763

(51) Int. Cl. A61K 38/19; A61K 31/727; A61K 38/48; A61K 9/14

(52) U.S. Cl. 424/85.1; 424/489; 514/56; 424/94.64; 514/12

(57) ABSTRACT

The present invention provides a polysaccharide-functionalized nanoparticle, a drug delivery system for controlled release comprising the nanoparticle and a preparation method thereof. In particular, the nanoparticle of the present invention comprises a core of a biodegradable polymer, an outer hydrogel layer of a biocompatible polymer emulsifier and a polysaccharide physically bound to the core and the hydrogel layer, thus enabling to significantly enhance stability and controlled release of a protein drug such as a growth factor.

### TECHNICAL FIELD

The present invention provides a polysaccharide-functionalized nanoparticle, a drug delivery system for controlled release comprising the nanoparticle and the preparation method thereof, and in particular the nanoparticle herein comprises a core of a biodegradable polymer, an outer hydrogel layer of a biocompatible polymer emulsifier and a polysaccharide physically bound to the core and the hydrogel layer, thus enabling to remarkably enhance the effects of stability and controlled release of a protein drug such as a growth factor.

### RELATED PRIOR ART

Therapeutic proteins or peptides such as growth factors and hormones have a very short half-life in a human body and are easily denatured at the hydrophilic-hydrophobic interface. Thus, it is very difficult to develop an efficient drug delivery system for controlled or sustained release of the therapeutic proteins as compared to that of a hydrophobic synthetic drug. Therefore, the development of microparticles or nanoparticles for delivering proteins has been mainly focused during the past decade on how to load a protein drug into a particle without much deterioration in its activity.

However, as disclosed in the recently issued U.S. Pat. Nos. 6,586,011 and 6,616,944, the problem of the activity deterioration has not been solved yet and a great deal of effort and time would be required to develop a polymer with secured stability to prevent the deterioration in its activity.

Further, U.S. Pat. No. 5,019,400 discloses a process of preparing micropheres for delivering proteins by spraying biocompatible poly(DL-lactide-co-glycolide) (‘PLGA’, hereinafter) into a very cold refrigerant. However, this process has a serious drawback of the deterioration in activity of protein drug due to the hydrophobicity of PLGA and an organic solvent used for dissolving PLGA.

Moreover, U.S. Pat. No. 6,586,011 discloses a process of preparing nanoparticles by spraying a mixture of biocompatible polyvinyl alcohol (‘PVA’, hereinafter) and plasminogen activators. However, this process shows a serious problem in the stability of protein because of a cross-linking agent.

Furthermore, U.S. Pat. No. 6,616,944 discloses a process comprising steps of introducing to PLGA polymer a functional group capable of forming an ionic bond with a protein and loading a protein drug to provide a protein drug-nanoparticle composite. However, this process also has a serious problem of causing polymer deformation and protein denaturation when forming the polymer-protein composite.

Meanwhile, in most of the drug delivery systems for controlled release of protein drugs developed until quite recently, the target material is just diffused into hydrogel for controlled release. However, these systems are also shown not very advantageous in that the initial burst of the drug is too high for the drug to be formulated to be released for a relatively long period of time.

### SUMMARY

The present inventors have made extensive and intensive researches to solve the aforementioned problems and finally found that a polysaccharide-functionalized nanoparticle herein, which comprises (a) a hydrophobic core of comprising a biodegradable polymer, (b) a hydrophilic surface hydrogel layer of comprising a biocompatible polymer emulsifier and (c) a polysaccharide physically bound to the core and the layer, is very effective in stabilizing the protein drug, decreasing an initial burst and prolong the release time.

Therefore, the present invention aims to provide a nanoparticle having a polysaccharide-functionalized hydrogel layer, which is excellent in stabilizing a protein drug and controlling an initial burst and a release time. The present invention also aims to provide a drug delivery system for controlled release, which comprises the nanoparticle herein, along with a preparation method thereof.

Other objects or advantages of the present invention are better understood by referring to the following Detailed Description and Drawings.

### DETAILED DESCRIPTION

According to one aspect of the present invention, there is provided a polysaccharide-functionalized nanoparticle comprising (a) a hydrophobic core of a biodegradable polymer, (b) a hydrophilic surface hydrogel layer of a biocompatible polymer emulsifier, and (c) a polysaccharide physically bound to the core and the layer.

According to an embodiment of the present invention, there is provided a polysaccharide-functionalized nanoparticle comprising (a) a hydrophobic core of at least one biodegradable polymer selected from the group consisting of poly(DL-lactide-co-glycolide), poly(lactic acid), poly(glycolic acid), poly(caprolactone), poly(valerolactone), poly(hydrobutyrate) and poly(hydroxyvalerate), (b) a hydrophilic surface hydrogel layer of at least one biocompatible polymer emulsifier selected from the group consisting of poloxamer, poloxamine, poly(vinyl alcohol) and poly(ethylene glycol) ether of alkyl alcohol, and (c) at least one polysaccharide selected from the group consisting of heparin, alginate, hyaruronic acid and chitosan, wherein the polysaccharide is physically bound to the core and the layer.

As used herein, the term of ‘a biodegradable polymer’ refers to a polymer that may degrade within an acceptable period of time in a physiological solution of pH 6-8, preferably body fluids or microorganisms in a human body.

Representative examples of the biodegradable polymer include but are not limited to PLGA of the following Formula 1, poly(lactic acid) (‘PLA’, hereinafter), poly(glycolic acid) (‘PGA’, hereinafter), poly(caprolactone) (‘PCL’, hereinafter), poly(valerolactone), poly(hydrobutyrate) (‘PHB’, hereinafter)), poly(hydroxyvalerate) and their combination. Further to the aforementioned polymers, any polymer may be used in the present invention only if it is sufficient for preparing a polysaccharide-functionalized nanoparticle when added into a biocompatible polymer emulsifier solution containing a polysaccharide. Preferably, FDA-approved PLGA may be used among these polymers.

A biodegradable polymer herein is preferred to have a weight average molecular weight (average Mw) of 5,000-100,000, more preferably 50,000-100,000, because the yield of nanoparticle production may be decreased and the stability of polysaccharide may be lowered due to the difficulty in molecular formation if the Mw is beyond the aforementioned range.

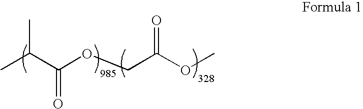

As used herein, the term of ‘a biocompatible polymer’ refers to a polymer having a tissue compatibility and a blood compatibility so that it does not cause a tissue necrosis nor a blood coagulation upon contact with tissue or blood, and ‘a biocompatible polymer emulsifier’ herein means a biocompatible polymer that is capable of emulsifying two or more separated phases.

Examples of the biocompatible polymer emulsifier herein include, without limitation, poloxamer, poloxamine, poly(vinyl alcohol), poly(ethylene glycol) ether of alkyl alcohol and their combination. Among these polymers, FDA-approved poloxamer is preferred.

Any polysaccharide may be used in the present invention only if it is capable of interaction with various proteins (or peptides) such as a growth factor or antithrombin III to inhibit the hydrolysis and maintain a three-dimensional structure of the protein, thus stabilizing the protein and enhancing the biological activity.

Examples of the polysaccharide herein include but are not limited to heparin of Formula 2 below, alginate, hyaruronic acid, chitosan and their combination. Among these polysaccharides, heparin, an anionic polysaccharide approved by FDA as non-cytotoxic, is preferred.

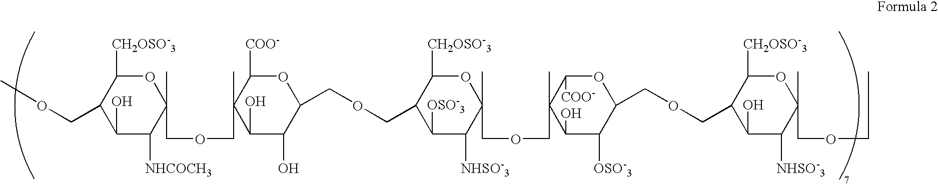

A nanoparticle herein comprises an inner core, an outer hydrogel layer and a polysaccharide that is physically bound to the core and the hydrogel layer. The polysaccharide forms a specific binding with a protein drug, thus being capable of stabilizing the protein drug and remarkably enhancing a controlled or sustained release by decreasing an initial release of the drug.

As used herein, the expressions of “physically bound” or like this refer to any kind of physical bindings induced by physical process without any chemical reaction, and the examples of the physical bindings include without limitation an adsorption, a coheison, an entanglement and an entrapment.

The nanoparticles herein are biocompatible only if the each ingredient is biocompatible because the polysaccharide is physically bound to a hydrogel layer and/or a core without any chemical reaction. In this regard, the nanoparticle and the drug delivery system according to the present invention are advantageous in terms of biocompatibility.

The nanoparticle is preferred to have a diameter of 400 nm or less as considering the sterilization process may be preformed conveniently by using a sterile filter. The surface charge is preferred to be −40 mV or less for effective loading of protein into a hydrogel layer and/or a core. It is preferred that the polydispersity is 0.1 or less for a stable monodispersity distribution.

According to another aspect of the present invention, there is provided a drug delivery system for controlled release comprising (a) a nanoparticle according to the present invention and (b) an effective amount of a drug, wherein the drug is a protein drug that may form a specific binding with the polysaccharide.

As used herein, the term of ‘a specific binding’ refers to a specific binding between a drug and a polysaccharide to form a relatively stable composite. The specific binding may be a covalent bond or a non-covalent bond, and especially includes the polysaccharide-protein interaction that inhibits the hydrolysis and maintains a three-dimensional structure of the protein, thus stabilizing the protein and enhancing its biological activity.

As used herein, ‘a drug’ or ‘a protein drug’ refers to any kind of protein (or polypeptide) drug that can form a specific binding with a polysaccharide. Examples of the drug include but are not limited to a growth factor such as a vascular endothelial growth factor (‘VEGF’, hereinafter), a fibroblast growth factor (‘FGF’, hereinafter), a platelet—derived growth factor (‘PDGF’, hereinafter), a chemokine, an extracellular matrix protein and an antithrombin III.

According to still another aspect of the present invention, there is provided a process of preparing a polysaccharide-functionalized nanoparticle, which comprises (a) obtaining an organic solution by dissolving a biodegradable polymer in a solvent that is not cytotoxic at a low concentration, (b) obtaining an aqueous solution by dissolving a polysaccharide and a biocompatible polymer emulsifier in water, and (c) mixing the organic solution and the aqueous solution.

According to an embodiment, there is provided a process of preparing a polysaccharide-functionalized nanoparticle, which comprises (a) obtaining an organic solution by dissolving a biodegradable polymer in a solvent that is not cytotoxic at low concentration, wherein the biodegradable polymer is at least one selected from the group consisting of poly(DL-lactide-co-glycolide), poly(lactic acid), poly(glycolic acid), poly(caprolactone), poly(valerolactone), poly(hydrobutyrate) and poly(hydroxyvalerate), and wherein the organic solvent is at least one selected from the group consisting of dimethylsufoxide, tetraglycol, ethyl lactate and ethanol; (b) obtaining an aqueous solution by dissolving a polysaccharide and an biocompatible polymer emulsifier in water, wherein the polysaccharide is at least one selected from the group consisting of heparin, alginate, hyaruronic acid and chitosan, and wherein the biocompatible polymer emulsifier is at least one selected from the group consisting of poloxamer, poloxamine, poly(vinyl alcohol) and poly(ethylene glycol) ether of alkyl alcohol; and (c) mixing dispersing the organic solution by adding it to the aqueous solution.

As used herein, “a solvent that is not cytotoxic at a low concentration” refers to a solvent that may remain within a nanoparticle and has been reported to non-cytotoxic at low concentration. Representative examples of the solvent include but are not limited to dimethylsulfoxide (‘DMSO’ hereinafter) or tetraglycol, both of which are reported to be non-cytotoxic at a concentration of 10% (v/v) or lower.

Any “water” may be used in the present invention only if it is biocompatible and does not show any toxicity, and is not limited to distilled water. Further, any conventional method may be used to add and disperse/emulsify organic solvent in an aqueous solvent.

Organic solution containing biocompatible polymer emulsifier is dispersed in aqueous solution containing polysaccharide and forms nanoparticles. The polysaccharide is preferred to be added in an amount of 10 wt % or less based on the weight of the biocompatible polymer emulsifier as considering polydispersity and production yield of the nanoparticles.

Further, the biocompatible polymer emulsifier aqueous solution is preferred to be prepared at a concentration of 5% or less as considering the thickness of hydrogel layer and the viscosity of aqueous solution for effective formation of nanoparticles.

Furthermore, the volume of the organic solvent is preferred to be, without any limitation, 10% or less based on the volume of the aqueous solution considering the amount of the organic solvent remaining in nanoparticles and cytotoxicity resulted therefrom.

According to a further aspect of the present invention, there is provided a process of preparing a drug delivery system for controlled release, which comprises (a) preparing a nanoparticle herein, (b) resuspending the nanoparticle, and (c) loading a drug into the resuspended nanoparticle.

### BRIEF DESCRIPTION OF DRAWINGS

FIG. 1 schematically shows a process of preparing heparin-functionalized PLGA nanoparticle.

FIG. 2 shows the sizes and the surface charges of the heparin-functionalized PLGA nanoparticles with varying amounts of heparin in an aqueous poloxamer solution.

FIG. 3 is a graph showing cumulative lysozyme releases from heparin-functionalized PLGA nanoparticles with different heparin contents.

FIG. 4 compares the concentrations of lysozyme released from the heparin-functionalized PLGA nanoparticles measured by the bioactivity on Micrococcus lysodeikticus cell walls using a lysozyme assay kit and the total protein concentration using a Micro BCA protein assay (n=3).

FIG. 5 shows cumulative releases of vascular endotherial growth factor (referred to as ‘VEGF’ hereinafter) from the heparin-functionalized PLGA nanoparticles with 4.7% w/w of heparin, where ▪ and □ respectively refer to nanoparticles loaded with 15.6 ng and 156 ng of VEGF, respectively, based on 1 mg of the nanoparticles.

### EXAMPLES

The present invention is described more specifically by the following Examples. Examples herein are meant only to illustrate the present invention, but in no way to limit the scope of the claimed invention.

The paper of “Biomaterials 27 (2006) 2621-2626” is incorporated by reference herein in their entirety for better understanding of the gist of the present invention, especially the experimental process herein.

### Comparative Example 1

### Preparation of Nanoparticle with Hydrophilic Hydrogel Layer

40 mg of PLGA was completely dissolved in 2 mL of dimethylsulfoxide, and this solution was slowly added in 30 mL of 5% aqueous poloxamer solution, thus providing nanoparticles.

### Examples 1-5

### Preparation of Nanoparticle with Heparin-Functionalized Hydrogel Layer

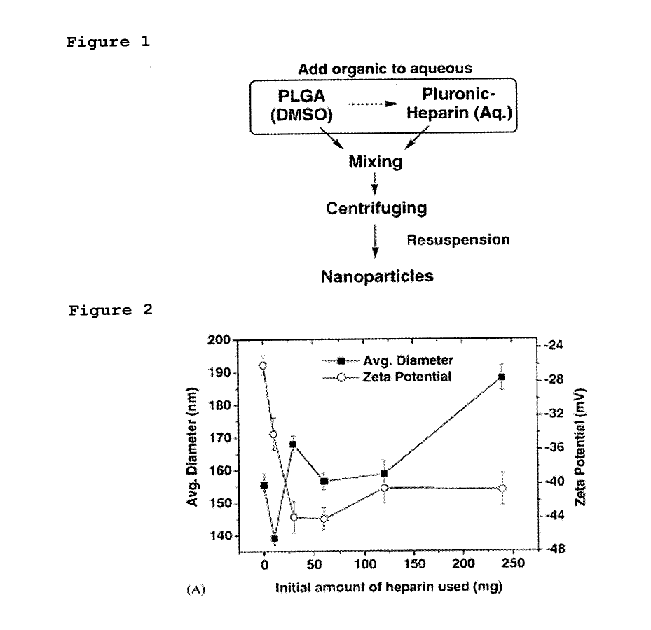

As shown in FIG. 1, 40 mg of PLGA was completely dissolved in 2 mL of dimethylsulfoxide, and this solution was slowly added in 30 mL of 5% aqueous poloxamer solution containing 10, 30, 60, 120 and 240 mg of heparin, respectively, thus providing heparin-functionalized nanoparticles.

### Comparative Example 2

### Preparation of Nanoparticle Loaded with Lysozyme

After preparing nanoparicles obtained in Comparative Example 1, remaining poloxamer and dimethylsulfoxide were removed by performing high-speed centrifugation and separating supernatant liquid. Thus obtained nanoparticles were resuspended in distilled water or PBS (phosphate buffered saline) solution (pH 7.4), and loaded with 1 mg of lysozyme by admixing the resuspended nanoparticles with 0.1 mL of PBS containing 1 mg of lysozyme, followed by incubation at 4° C. overnight with gentle rotation.

### Examples 6 & 7

### Preparation of Heparin-Functionalized Nanoparticle Loaded with Lysozyme

After preparing heparin-functionalized nanoparticles in Examples 2 & 4, remaining heparin, poloxamer and dimethylsulfoxide were removed by performing high-speed centrifugation and separating a supernatant liquid. Thus obtained nanoparticles were resuspended in distilled water or PBS solution, and loaded with 1 mg of lysozyme by mixing the resuspended nanoparticles with 0.1 mL of PBS containing 1 mg of lysozyme, followed by incubation at 4° C. overnight with gentle rotation.

### Examples 8 & 9

### Preparation of Heparin-Functionalized Nanoparticle Loaded with VEGF

The heparin-functionalized nanoparticles prepared in Example 4 were loaded with VEGF as described in Examples 6 & 7. One group of nanoparticles was loaded with 15.6 ng of VEGF and another group was loaded with 156 ng of VEGF based on 1 mg of the nanoparticles.

### Experimental Example 1

### Observation of Size, Surface Charge, Contents and Polydispersity of Nanoparticle

The size and the surface charge of the prepared nanoparticles were measured according to dynamic light scattering method and electrophoretic light scattering method, respectively, by using ELS-8000 (Otsuka Electronics Co., Japan)

As shown in FIG. 2, the size increased from 123.1±2.0 nm to 188.1±3.9 and the surface charge varied from −26.0±1.1 mV to −44.4±1.2 mV with the increase of heparin amount in an aqueous poloxamer solution. As the heparin carries a strong negative charges and relatively higher negative value in surface charge means that a higher amount of heparin exists on the surface of the nanoparticles.

Dry weight of the nanoparticles was calculated after freeze drying the nanoparticles. A partial amount of heparin in hydrogel layer and the total amount in nanoparicles were calculated through an anti-factor Xa analysis (C. Chauvierre et al., Biomaterials 25 (2004) 3081-3086) by using particle-state nanoparticles and nanoparticle solution, respectively. The ratio of PLGA to poloxamer in nanoparticles was finally obtained by performing 1 H NMR analysis, to provide mass ratio of each ingredient as shown in Table 1.

As shown in Table 1, most of physically-bound heparin was verified to exist in a surface layer. Further, high-speed centrifugation removed non-bound heparin including heparin that was just dispersed in hydrogel layer, which shows that the heparin exiting in a surface layer is physically bound to the hydrogel surface layer comprising poloxamer and/or a core. It is assured that poloxamer is stabilized by a hydrophobic interaction with PLGA and heparin is fixed to the surface layer via hydrophilic interaction between the carboxylic groups in heparin and poly(ethylene glycol) in poloxamer.

Table 1 provides contents of each ingredients in nanoparticles prepared in Comparative Example 1 and Examples 2 and 4.

TABLE 1
Contents of Each Ingredients in Nanoparticles

| Heparin | | | Heparin | | Total Amount | |
|---|---|---|---|---|---|---|
| in solution | PLGA | Poloxamer | in Surface Layer | | of Heparin | |
| 0 mg | 36.8 ± 1.6 mg | 13.8 ± 0.6 mg | 0.0 | mg | 0.0 | mg |
| | (72.7%) | (27.3%) | (0.0%) | | (0.0%) | |
| 30 mg | 34.7 ± 0.9 mg | 13.3 ± 0.4 mg | 0.94 ± 0.04 | mg | 1.20 ± 0.04 | mg |
| | (70.6%) | (27.0%) | (1.9%) | | (2.4%) | |
| 120 mg | 29.0 ± 1.8 mg | 12.3 ± 0.8 mg | 1.71 ± 0.09 | mg | 2.01 ± 0.10 | mg |

| | | | |
|---|---|---|---|
| (66.9%) | (28.4%) | (4.0%) | (4.7%) |

Table 1 shows that the amount of heparin in hydrogel surface layer increases with the increase of the amount of heparin in an aqueous poloxamer solution. However, the aqueous solution is preferred to contain heparin in the concentration of 120 mg/2 mL or less considering polydispersity and production yield of nanoparticles.

### Experimental Example 2

### Observation of In Vitro Controlled Release and Stabilizing Effect of Lysozime

The following in vitro experiment was performed to verify the controlled release of proteins and the stability effect with regard to the nanoparticles prepared in Comparative Example 2 and Examples 6 and 7.

The resuspended heparin-functionalized nanoparticles were mixed with 0.1 mL of PBS solution containing 1 mg of lysozyme, and then incubated at 4° C. overnight with gentle rotation, thus providing 43.4 nanoparticles loaded with 1 mg of lysozyme.

After 6 mL of the lysozyme-loaded nanoparticle suspension was put into a dialysis tube (MWCO 500 k), the released lysozyme was collected by using a large amount of PBS solution under the infinite dilution condition. The amount of the collected lysozyme was quantified according to the Micro BCA protein quantification. PBS used for collecting lysozyme was replaced with new one every day, and the sample was stored at 4° C. until the protein quantification was performed.

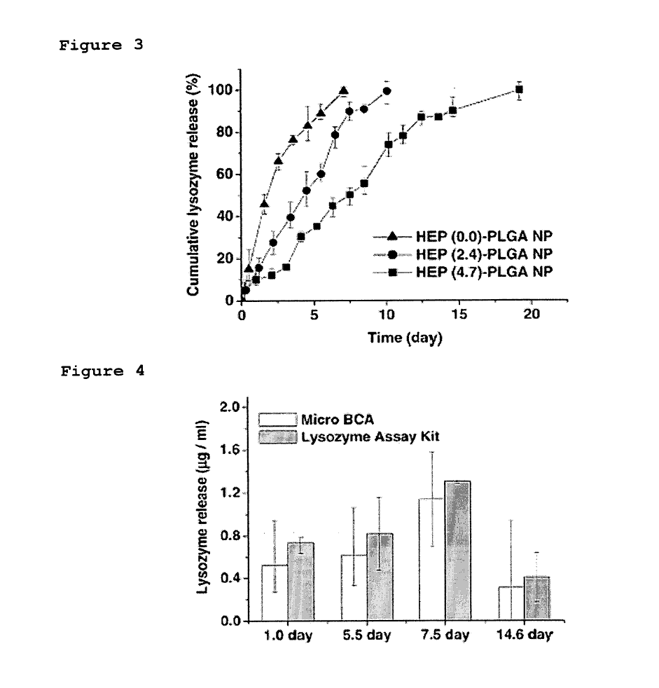

The result of the release experiments is provided in FIG. 3, which is a graph showing cumulative lysozyme releases from heparin-functionalized PLGA nanoparticles with different heparin contents. A nanoparticle with no heparin releases about two thirds amount of drug within 3 days, while the release lasted for up to 19 days without an initial burst in a nanoparticle with 4.7 wt % of heparin. This result shows that the increase in heparin amount in turn enhances the effect of the controlled release.

Biological activity of the released lysozyme was observed and the result is provided in FIG. 4, which shows that the loaded protein drug was stabilized by the heparin fixed to the hydrogel surface layer.

### Experimental Example 3

### Observation of In Vitro Controlled Release and Stabilizing Effect of VEGF

The following in vitro experiment was performed to verify the controlled release of proteins and the stability effect with regard to the nanoparticles prepared in Example 8.

Both (i) one-third and (ii) one-tenth of resuspended heparin-functionalized nanoparticles were mixed with (i) 25 mL of PBS solution containing 250 ng of VEGF and (ii) 75 mL of PBS solution containing 750 ng of VEGF, respectively, and then incubated at 4° C. overnight with gentle rotation, thus providing (i) 14.4 mg of heparin-functionalized nanoparticle loaded with 250 ng of VEGF and (ii) 4.3 mg of heparin-functionalized nanoparticle loaded with 750 ng of VEGF, respectively.

After 0.2 mL of the VEGF-loaded nanoparticle suspension was put into a dialysis tube (MWCO 500 k), the released VEGF was collected by using a large amount of PBS solution under the infinite dilution condition. The amount of the collected VEGF was quantified by using ELISA analysis (enzyme-linked immunosorbent assay, ELISA. The sample was stored at −30° C. until the protein quantification was performed.

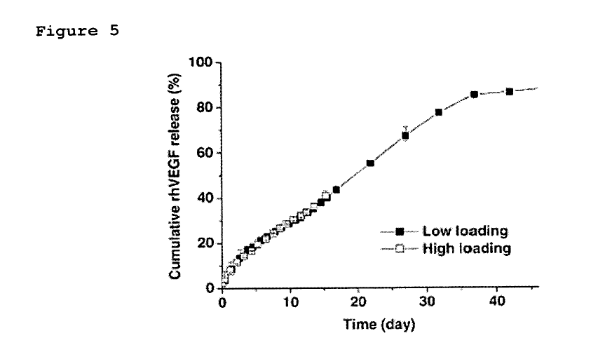

The result of the release experiments is provided in FIG. 5, which is a graph showing cumulative VEGF release from heparin-functionalized PLGA nanoparticles having 4.7 wt % of heparin. The nanoparticle loaded with 15.6 ng VEGF in 1 mg of the nanoparticles released drugs up to 85% of the initial amount for 37 days without an initial burst.

Meanwhile, the experiment for the ten times of loading amount (156 ng of VEGF in 1 mg of nanoparticles) was performed under the same condition, and the result is provided in FIG. 5. The cumulative amount of VEGF release with the time was observed to be the same as in the Experimental Example 2, which shows that the controlled release of the drug is induced by the specific binding with the heparin that is physically bound to the hydrogel layer and/or the core.

As set forth in the above, the drug delivery system herein released protein while the activity is maintained and the specific binding between the heparin and the drug induced the controlled release. Thus, the present invention may be usefully applied to development of drug delivery system or a method of loading protein drug without any deterioration in activity.

What is claimed is:

1. A polysaccharide-functionalized nanoparticle comprising:
(a) a hydrophobic core comprising a biodegradable polymer,
(b) a hydrophilic surface hydrogel layer comprising a biocompatible polymer emulsifier, and
(c) a polysaccharide physically bound to the core and the layer.

2. A polysaccharide-functionalized nanoparticle comprising:
(a) a hydrophobic core comprising at least one biodegradable polymer selected from the group consisting of poly(DL-lactide-co-glycolide), poly(lactic acid), poly(glycolic acid), poly(caprolactone), poly(valerolactone), poly(hydrobutyrate) and poly(hydroxyvalerate),
(b) a hydrophilic surface hydrogel layer comprising at least one biocompatible polymer emulsifier selected from the group consisting of poloxamer, poloxamine, poly(vinyl alcohol) and poly(ethylene glycol) ether of alkyl alcohol, and
(c) at least one polysaccharide selected from the group consisting of heparin, alginate, hyaruronic acid and chitosan, wherein the polysaccharide is physically bound to the core and the layer.

3. The polysaccharide-functionalized nanoparticle of claim 2, wherein the biodegradable polymer has a weight average molecular weight of 5,000-100,000.

4. The polysaccharide-functionalized nanoparticle of claim 3, wherein the nanoparticle has a diameter of 400 nm or less, a surface charge of −40 mV or less and a polydispersity of 0.1 or less.

5. The polysaccharide-functionalized nanoparticle of claim 4, wherein the biodegradable polymer is poly(DL-lactide-co-glycolide), the biocompatible polymer emulsifier is poloxamer and the polysaccharide is heparin.

6. A drug delivery system for controlled release comprising:
(a) a nanoparticle of claim 1, and
(b) an effective amount of a drug,
wherein the drug is a protein drug that may form a specific binding with the polysaccharide.

7. A drug delivery system for controlled release comprising:
(a) a nanoparticle of claim 2, and
(b) an effective amount of a drug,
wherein the drug is a protein drug that may form a specific binding with the polysaccharide.

8. The drug delivery system of claim 7, wherein the drug is at least one selected from the group consisting of a growth factor, a chemokine, an extracellular matrix protein and an antithrombin III.

9. The drug delivery system of claim 8, wherein the biodegradable polymer has a weight average molecular weight of 5,000-100,000.

10. The drug delivery system of claim 9, wherein the nanoparticle has a diameter of 400 nm or less, a surface charge of −40 mV or less and a polydispersity of 0.1 or less.

11. The drug delivery system of claim 10, wherein the biodegradable polymer is poly(DL-lactide-co-glycolide), the biocompatible polymer emulsifier is poloxamer and the polysaccharide is heparin.

12. A process of preparing a polysaccharide-functionalized nanoparticle, the process comprising:
(a) obtaining an organic solution by dissolving a biodegradable polymer in a solvent that is not cytotoxic at a low concentration,
(b) obtaining an aqueous solution by dissolving a polysaccharide and an biocompatible polymer emulsifier in water, and
(c) mixing the organic solution and the aqueous solution.

13. A process of preparing a polysaccharide-functionalized nanoparticle, the process comprising:
(a) obtaining an organic solution by dissolving a biodegradable polymer in a solvent that is not cytotoxic at a low concentration,
wherein the biodegradable polymer is at least one selected from the group consisting of poly(DL-lactide-co-glycolide), poly(lactic acid), poly(glycolic acid), poly(caprolactone), poly(valerolactone), poly(hydrobutyrate) and poly(hydroxyvalerate), and
wherein the organic solvent is at least one selected from the group consisting of dimethylsufoxide, tetraglycol, ethyl lactate and ethanol;
(b) obtaining an aqueous solution by dissolving a polysaccharide and a biocompatible polymer emulsifier in water,
wherein the polysaccharide is at least one selected from the group consisting of heparin, alginate, hyaruronic acid and chitosan, and
wherein the biocompatible polymer emulsifier is at least one selected from the group consisting of poloxamer, poloxamine, poly(vinyl alcohol) and poly(ethylene glycol) ether of alkyl alcohol; and
(c) mixing the organic solution and the aqueous solution.

14. The process of claim 13, wherein (c) mixing is performed by slowly adding the organic solution into the aqueous solution with vigorous stirring, and wherein the polysaccharide is used in the amount of 10 wt % or less with reference to that of the polymer emulsifier and the organic solution is used 10 vol % with reference to that of the aqueous solution.

15. The process of claim 14, wherein the biodegradable polymer has a weight average molecular weight of 5,000-100,000.

16. The process of claim 15, wherein the nanoparticle has a diameter of 400 nm or less, a surface charge of −40 mV or less and a polydispersity of 0.1 or less.

17. A process of preparing a drug delivery system for controlled release, the process comprising:
(a) preparing a nanoparticle according to claim 12,
(b) resuspending the nanoparticle into a PBS solution, and
(c) loading a protein drug into the resuspended nanoparticle.

18. The process of claim 17, wherein the protein drug is at least one selected from the group consisting of a growth factor, a chemokine, an extracellular matrix protein and an antithrombin III.
